(19) United States
(12) Patent Application Publication
(10) Pub. No.: US 2025/0002454 A1
(43) Pub. Date: Jan. 2, 2025

(54) ELECTROLYTE ADDITIVES FOR ZINC BATTERIES

(71) Applicant: Octet Scientific, Inc., Cleveland, OH (US)

(72) Inventors: Onas James BOLTON, Papper Pike, OH (US); Emily Janine DICKENS, Cleveland, OH (US)

(21) Appl. No.: 18/273,863

(22) PCT Filed: Jan. 28, 2022
(86) PCT No.: PCT/US2022/014435

§ 371 Date: Jul. 24, 2023

Related U.S. Application Data
(60) Provisional application No. 63/145,294, filed on Feb. 3, 2021.

(51) Int. Cl. C07C 323/12; C07C 323/25; H01M 12/02

(52) U.S. Cl. CPC C07C 323/12; C07C 323/25; H01M 12/02; H01M 2300/0005

(57) ABSTRACT

Provided herein are novel zinc-battery electrolyte additive chemicals with surprising advantageous properties such as, but not limited to, dendrite prevention and efficiency promotion.

### CROSS-REFERENCE TO RELATED APPLICATIONS

This application claims the benefit of, and priority to, U.S. Provisional Patent Application No. 63/145,294, filed Feb. 3, 2021, the entire contents of which are herein incorporate by reference in their entirety for all purpose.

### STATEMENT OF GOVERNMENT RIGHTS

This invention was made with government support under SBIR 2013880 awarded by the National Science Foundation. The government has certain rights in the invention.

### FIELD

The present disclosure concerns zinc-based rechargeable batteries, such as, but not limited to, zinc, zinc-lithium, zinc-carbon, zinc-chloride, zinc-bromide, zinc-air, and other related zinc-anode-including batteries.

### BACKGROUND

Dendrite growth on zinc anodes is a major cause of failure and poor performance for zinc-batteries. One method of controlling and mitigating if not preventing dendrite formation includes using battery electrolyte additive chemicals. See, e.g., US Patent Publication No. 2020/0243909, which published Jul. 30, 2020, and is titled ZINC BATTERY ELECTROLYTE ADDITIVE, the entire contents of which are herein incorporated by reference in its entirety for all purposes.

What is needed are novel compositions and methods for controlling and preventing dendrite formation in zinc-batteries.

### SUMMARY

Provided herein are novel zinc-battery electrolyte additive chemicals with surprising advantageous properties such as, but not limited to, dendrite prevention and efficiency promotion. When about 0.005 weight percent (wt %) to about 25 wt % of the electrolyte, relative to the total mass of the electrolyte, includes the novel zinc-battery electrolyte additive chemicals set forth herein, the zinc-battery demonstrate unexpectedly improved performance during charging, discharging, and storage.

In one example, set forth herein is an electrolyte additive, having the following structure:

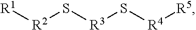

in which R¹ and R⁵ are, independently in each instance, selected from —OH, —NR⁶R⁷; —NR⁶R⁷R⁸; —O—R⁶; —O—CR⁶R⁷R⁸; —O—C(O)—R⁸; and —C(O)OH; R² and R⁴ are, independently in each instance, selected from an optionally substituted alkylene and an optionally substituted heteroalkylene, wherein the alkylene and heteroalkylene, independently in each instance, has 1 to 10 carbon atoms; R³ is absent or is selected from an optionally substituted alkylene and an optionally substituted heteroalkylene, wherein the alkylene and heteroalkylene, independently in each instance, has 1 to 10 carbon atoms; R⁶ and R⁷ are, independently in each instance, selected from H, alkyl, heteroalkyl, aryl, heteroaryl, alkyl-aryl, and alkyl-heteroaryl; wherein the alkyl, heteroalkyl, aryl, heteroaryl, alkyl-aryl, and alkyl-heteroaryl are unsubstituted; or R⁶ and R⁷, together with the atom to which they are attached, form an optionally substituted cycloalkyl or heterocycloalkyl having 4 to 6 carbon atoms; R⁸ is absent or is selected from H, alkyl, heteroalkyl, aryl, heteroaryl, alkyl-aryl, and alkyl-heteroaryl; wherein the alkyl, heteroalkyl, aryl, heteroaryl, alkyl-aryl, and alkyl-heteroaryl are unsubstituted; wherein electrolyte additive is present in an electrolyte at a concentration equal to, or greater than, 0.005 weight percent (wt %) to less than, or equal to, 25 wt %. Optionally substituted substituents are unsubstituted unless explicitly stated otherwise.

In another example, set forth herein is a process for making a zinc battery, comprising contacting an electrolyte having an electrolyte additive described herein with a zinc-battery electrode.

In another example, set forth herein is a method of using a zinc battery, comprising electrochemically cycling a zinc-battery comprising an electrolyte having an electrolyte set forth herein.

In another example, set forth herein is a method of using a zinc battery, comprising charging a zinc-battery comprising an electrolyte having an electrolyte additive described herein to at least −1 V (relative to Ag/AgCl).

### BRIEF DESCRIPTIONS OF THE DRAWINGS

FIG. 1A shows an optical image a zinc-battery negative electrode described in Example 1.

FIG. 1B shows an optical image a zinc-battery negative electrode described in Example 1.

FIG. 2A shows an optical image a zinc-battery negative electrode described in Example 2.

FIG. 2B shows an optical image a zinc-battery negative electrode described in Example 2.

FIG. 3 shows an optical image a zinc-battery negative electrode described in Example 3.

FIG. 4 shows an optical image a zinc-battery negative electrode described in Example 4.

FIG. 5 shows an optical image a zinc-battery negative electrode described in Example 5.

FIG. 6 shows an optical image a zinc-battery negative electrode described in Example 6.

### DETAILED DESCRIPTION

The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the disclosure herein is not intended to be limited to the embodiments presented, but are to be accorded their widest scope consistent with the principles and novel features disclosed herein.

All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

Please note, if used, the labels left, right, front, back, top, bottom, forward, reverse, clockwise and counter clockwise have been used for convenience purposes only and are not intended to imply any particular fixed direction. Instead, they are used to reflect relative locations and/or directions between various portions of an object.

### I. DEFINITIONS

As used herein, the singular forms “a,” “an” and “the” include plural referents unless the context clearly dictates otherwise.

As used herein, the term “about,” when qualifying a number, e.g., 15% w/w, refers to the number qualified and optionally the numbers included in a range about that qualified number that includes ±10% of the number. For example, about 15% w/w includes 15% w/w as well as 13.5% w/w, 14% w/w, 14.5% w/w, 15.5% w/w, 16% w/w, or 16.5% w/w.

As used herein, “selected from the group consisting of” refers to a single member from the group, more than one member from the group, or a combination of members from the group. A member selected from the group consisting of A, B, and C includes, for example, A only, B only, or C only, as well as A and B, A and C, B and C, as well as A, B, and C.

As used herein, “alkyl” refers to a monovalent and saturated hydrocarbon radical moiety. Alkyl is optionally substituted and can be linear, branched, or cyclic, i.e., cycloalkyl. Alkyl includes, but is not limited to, those having 1-10 carbon atoms, i.e., C₁₋₁₀ alkyl; Examples of alkyl moieties include, but are not limited to methyl, ethyl, n-propyl, i-propyl, n-butyl, s-butyl, t-butyl, i-butyl, a pentyl moiety, a hexyl moiety, cyclopropyl, cyclobutyl, cyclopentyl, and cyclohexyl.

As used herein, “heteroalkyl” refers to an alkyl in which one or more carbon atoms are replaced by heteroatoms. Suitable heteroatoms include, but are not limited to, nitrogen, oxygen, and sulfur atoms. Heteroalkyl is optionally substituted. Examples of heteroalkyl moieties include, but are not limited to, aminoalkyl, sulfonylalkyl, sulfinylalkyl. Examples of heteroalkyl moieties also include, but are not limited to, methylamino and methylsulfonyl.

As used herein, “heteroaryl” refers to a monovalent moiety that is a radical of an aromatic compound wherein the ring atoms contain carbon atoms and at least one oxygen, sulfur, nitrogen, or phosphorus atom. Examples of heteroaryl moieties include, but are not limited to those having 5 to 20 ring atoms; 5 to 15 ring atoms; and 5 to 10 ring atoms. Heteroaryl is optionally substituted unless explicitly stated otherwise.

As used herein, “aryl” refers to a monovalent moiety that is a radical of an aromatic compound wherein the ring atoms are carbon atoms. Aryl is optionally substituted and can be monocyclic or polycyclic, e.g., bicyclic or tricyclic. Examples of aryl moieties include, but are not limited to those having 6 to 20 ring carbon atoms, i.e., C₆₋₂₀ aryl; 6 to 15 ring carbon atoms, i.e., C₆₋₁₅ aryl, and 6 to 10 ring carbon atoms, i.e., C₆₋₁₀ aryl. Examples of aryl moieties include, but are limited to, phenyl, naphthyl, fluorenyl, azulenyl, anthryl, phenanthryl, and pyrenyl.

As used herein, “heteroaryl” refers to a monovalent moiety that is a radical of an aromatic compound wherein the ring atoms contain carbon atoms and at least one oxygen, sulfur, nitrogen, or phosphorus atom. Examples of heteroaryl moieties include, but are not limited to those having 5 to 20 ring atoms; 5 to 15 ring atoms; and 5 to 10 ring atoms. Heteroaryl is optionally substituted unless explicitly stated otherwise.

As used herein, “alkylene” refers to a divalent moiety of an alkyl compound. Alkylene may have from 2 to 10 carbon atoms, e.g., C₂alkylene, C₃alkylene, C₄alkylene, C₅alkylene, C₆alkylene, C₇alkylene, C₈alkylene, C₉alkylene, or C₁₀alkylene. Examples of alkylene moieties include, but are not limited to methylene, ethylene, propylene, butylene, pentylene, and hexylene.

As used herein, “heteroalkylene” refers to a divalent moiety of a heteroalkyl compound. Heteroalkylene may have from 2 to 10 carbon atoms, e.g., C₂heteroalkylene, C₃heteroalkylene, C₄heteroalkylene, C₅heteroalkylene, C₆heteroalkylene, C₇heteroalkylene, C₈heteroalkylene, C₉heteroalkylene, or C₁₀heteroalkylene.

As used herein, “alky-aryl” refers to an alkyl group, as defined herein, which is substituted with an aryl group, as defined herein.

As used herein, “alky-heteroaryl” refers to an alkyl group, as defined herein, which is substituted with an heteroaryl group, as defined herein.

As used herein, “heterocycloalkyl” refers to a cycloalkyl in which one or more carbon atoms are replaced by heteroatoms. Suitable heteroatoms include, but are not limited to, nitrogen, oxygen, and sulfur atoms. Heterocycloalkyl is optionally substituted. Examples of heterocycloalkyl moieties include, but are not limited to, morpholinyl, piperidinyl, tetrahydropyranyl, pyrrolidinyl, imidazolidinyl, oxazolidinyl, thiazolidinyl, dioxolanyl, dithiolanyl, oxanyl, or thianyl.

As used herein, “optionally substituted,” when used to describe a radical moiety, e.g., optionally substituted alkyl, means that such moiety is optionally bonded to one or more substituents. Examples of such substituents include, but are not limited to halo, cyano, nitro, haloalkyl, azido, epoxy, optionally substituted heteroaryl, optionally substituted heterocycloalkyl,

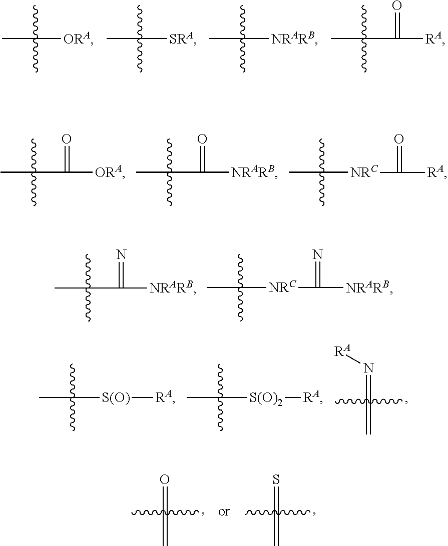

wherein R^{A}, R^{B}, and R^{C} are, independently at each occurrence, a hydrogen atom, alkyl, alkenyl, alkynyl, aryl, alkaryl, aralkyl, heteroalkyl, heteroaryl, or heterocycloalkyl, or R^{A} and R^{B}, together with the atoms to which they are bonded, form a saturated or unsaturated carbocyclic ring, wherein the ring is optionally substituted and wherein one or more ring atoms is optionally replaced with a heteroatom. In certain embodiments, when a radical moiety is optionally substituted with an optionally substituted heteroaryl, optionally substituted heterocycloalkyl, or optionally substituted saturated or unsaturated carbocyclic ring, the substituents on the optionally substituted heteroaryl, optionally substituted heterocycloalkyl, or optionally substituted saturated or unsaturated carbocyclic ring, if they are substituted, are not substituted with substituents which are further optionally substituted with additional substituents. In some embodiments, when a group described herein is optionally substituted, the substituent bonded to the group is unsubstituted unless otherwise specified.

### II. ELECTROLYTES

Set forth herein are new electrolytes and electrolyte additives. These electrolytes and electrolyte additives are useful as electrolytes in zinc batteries. Zinc batteries includes zinc-air batteries as well as other types of zinc batteries. In some examples the electrolytes contemplated herein are neutral (with respect to pH). In some other examples the electrolytes contemplated herein are acidic (with respect to pH). One example of an acidic electrolyte is a zinc-bromine electrolyte. In yet other examples the electrolytes contemplated herein are basic (with respect to pH). One example of a basic electrolyte is a zinc-air battery. In addition, the electrolytes set forth herein may be used in zinc-manganese oxide batteries. The electrolytes set forth herein may be used in nickel-zinc batteries. Furthermore, the electrolytes set forth herein may be used in silver-zinc batteries. Additionally, the electrolytes set forth herein may be used in zinc-lithium batteries.

In one example, set forth herein is an electrolyte additive, having the following structure:

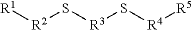

In some examples, including any of the foregoing, R¹ and R⁵ are, independently in each instance, selected from —OH, —NR⁶R⁷; —NR⁶R⁷R⁸; —O—R⁶; —O—CR⁶R⁷R⁸; —O—C(O)—R⁸; and —C(O)OH. In certain examples R¹ is —OH. In certain other examples R¹ is —NR⁶R⁷. In some examples R¹ is —NR⁶R⁷R⁸. In some other examples R¹ is —O—R⁶. In certain examples R¹ is —O—CR⁶R⁷R⁸. In certain other examples R¹ is —O—C(O)—R⁸. In some examples R¹ is —C(O)OH. In certain examples R⁵ is —OH. In certain other examples R⁵ is —NR⁶R⁷. In some examples R⁵ is —NR⁶R⁷R⁸. In some other examples R⁵ is —O—R⁶. In certain examples R⁵ is —O—CR⁶R⁷R⁸. In certain other examples R⁵ is —O—C(O)—R⁸. In some examples R⁵ is —C(O)OH. In some of these examples, R² and R⁴ are, independently in each instance, selected from an optionally substituted alkylene and an optionally substituted heteroalkylene, wherein the alkylene and heteroalkylene, independently in each instance, has 1 to 10 carbon atoms. In some of these examples, R² is optionally substituted C₂alkylene. In some of these examples, R² is optionally substituted C₃alkylene. In some of these examples, R² is optionally substituted C₄alkylene. In some of these examples, R² is optionally substituted C₅alkylene. In some of these examples, R² is optionally substituted C₆alkylene. In some of these examples, R² is optionally substituted C₇alkylene. In some of these examples, R² is optionally substituted C₈alkylene. In some of these examples, R² is optionally substituted C₉alkylene. In some of these examples, R² is optionally substituted C₁₀alkylene. In some of these examples, R⁴ is optionally substituted C₂alkylene. In some of these examples, R⁴ is optionally substituted C₃alkylene. In some of these examples, R⁴ is optionally substituted C₄alkylene. In some of these examples, R⁴ is optionally substituted C₅alkylene. In some of these examples, R⁴ is optionally substituted C₆alkylene. In some of these examples, R⁴ is optionally substituted C₇alkylene. In some of these examples, R⁴ is optionally substituted C₈alkylene. In some of these examples, R⁴ is optionally substituted C₉alkylene. In some of these examples, R⁴ is optionally substituted C₁₀alkylene. In some of these examples, R² is optionally substituted C₂heteroalkylene. In some of these examples, R² is optionally substituted C₃heteroalkylene. In some of these examples, R² is optionally substituted C₄heteroalkylene. In some of these examples, R² is optionally substituted C₅heteroalkylene. In some of these examples, R² is optionally substituted C₆heteroalkylene. In some of these examples, R² is optionally substituted C₇heteroalkylene. In some of these examples, R² is optionally substituted C₈heteroalkylene. In some of these examples, R² is optionally substituted C₉heteroalkylene. In some of these examples, R² is optionally substituted C₁₀heteroalkylene. In some of these examples, R⁴ is optionally substituted C₂heteroalkylene. In some of these examples, R⁴ is optionally substituted C₃heteroalkylene. In some of these examples, R⁴ is optionally substituted C₄heteroalkylene. In some of these examples, R⁴ is optionally substituted C₅heteroalkylene. In some of these examples, R⁴ is optionally substituted C₆heteroalkylene. In some of these examples, R⁴ is optionally substituted C₇heteroalkylene. In some of these examples, R⁴ is optionally substituted C₈heteroalkylene. In some of these examples, R⁴ is optionally substituted C₉heteroalkylene. In some of these examples, R⁴ is optionally substituted C₁₀heteroalkylene. In some examples, R³ is absent or is selected from an optionally substituted alkylene and an optionally substituted heteroalkylene, wherein the alkylene and heteroalkylene, independently in each instance, has 1 to 10 carbon atoms. In certain of these examples, R³ is absent. In some of these examples, R³ is optionally substituted C₂heteroalkylene. In some of these examples, R³ is optionally substituted C₃heteroalkylene. In some of these examples, R³ is optionally substituted C₄heteroalkylene In some of these examples, R³ is optionally substituted C₅heteroalkylene. In some of these examples, R³ is optionally substituted C₆heteroalkylene. In some of these examples, R³ is optionally substituted C₇heteroalkylene. In some of these examples, R³ is optionally substituted C₈heteroalkylene. In some of these examples, R³ is optionally substituted C₉heteroalkylene. In some of these examples, R³ is optionally substituted C₁₀heteroalkylene. In some examples, R⁶ and R⁷ are, independently in each instance, selected from H, alkyl, heteroalkyl, aryl, heteroaryl, alkyl-aryl, and alkyl-heteroaryl; wherein the alkyl, heteroalkyl, aryl, heteroaryl, alkyl-aryl, and alkyl-heteroaryl are unsubstituted; or R⁶ and R⁷, together with the atom to which they are attached, form an optionally substituted cycloalkyl or heterocycloalkyl having 4 to 6 carbon atoms. In some examples, R⁶ is H. In some examples, R⁶ is alkyl. In some examples, R⁶ is heteroalkyl. In some examples, R⁶ is aryl. In some examples, R⁶ is heteroaryl. In some examples, R⁶ is alkyl-aryl. In some examples, R⁶ is alkyl-heteroaryl. In some examples, R⁷ is H. In some examples, R⁷ is alkyl. In some examples, R⁷ is heteroalkyl. In some examples, R⁷ is aryl. In some examples, R⁷ is heteroaryl. In some examples, R⁷ is alkyl-aryl. In some examples, R⁷ is alkyl-heteroaryl. In some examples, R⁶ and R⁷, together with the atom to which they are attached, form an optionally substituted cycloalkyl or heterocycloalkyl having 4 to 6 carbon atoms. In some other examples, R⁶ and R⁷, together with the atom to which they are attached, form an optionally substituted heterocyclic ring having 4 to 6 carbon atoms. In some examples, R⁶ and R⁷, together with the atom to which they are attached, form an optionally substituted cycloalkyl having 4 carbon atoms. In some other examples, R⁶ and R⁷, together with the atom to which they are attached, form an optionally substituted heterocycloalkyl having 4 carbon atoms. In some examples, R⁶ and R⁷, together with the atom to which they are attached, form an optionally substituted cycloalkyl having 5 carbon atoms. In some other examples, R⁶ and R⁷, together with the atom to which they are attached, form an optionally substituted heterocycloalkyl ring having 5 carbon atoms. In some examples, R⁶ and R⁷, together with the atom to which they are attached, form an optionally substituted cycloalkyl having 6 carbon atoms. In some other examples, R⁶ and R⁷, together with the atom to which they are attached, form an optionally substituted heterocycloalkyl having 6 carbon atoms. In some examples, the heterocycloalkyl is selected from azetidinyl, oxetanyl, pyrrolyl, furanyl, and pyridinyl, morpholinyl, piperidinyl, tetrahydropyranyl, pyrrolidinyl, imidazolidinyl, oxazolidinyl, thiazolidinyl, dioxolanyl, dithiolanyl, oxanyl, or thianyl. In some examples, R⁸ is absent or is selected from H, alkyl, heteroalkyl, aryl, heteroaryl, alkyl-aryl, and alkyl-heteroaryl; wherein the alkyl, heteroalkyl, aryl, heteroaryl, alkyl-aryl, and alkyl-heteroaryl are unsubstituted. In certain examples, R⁸ is absent. In other examples, R⁸ is selected from H, alkyl, heteroalkyl, aryl, heteroaryl, alkyl-aryl, and alkyl-heteroaryl; wherein the alkyl, heteroalkyl, aryl, heteroaryl, alkyl-aryl, and alkyl-heteroaryl are unsubstituted. In some of these examples, R⁸ is H. In some of these examples, R⁸ is alkyl. In certain of these examples, R⁸ is heteroalkyl. In some other examples, R⁸ is aryl. In some examples, R⁸ is heteroaryl. In some other examples, R⁸ is alkyl-aryl. In some examples, R⁸ is alkyl-heteroaryl. In certain examples, the electrolyte additive is present in an electrolyte at a concentration equal to, or greater than, about 0.005 weight percent (wt %) to about 25 wt %. In certain other examples, the electrolyte additive is present in an electrolyte at a concentration equal to, or greater than, 0.005 weight percent (wt %) to less than, or equal to, 25 wt %.

In some examples, including any of the foregoing, the electrolyte additive has the following structure:

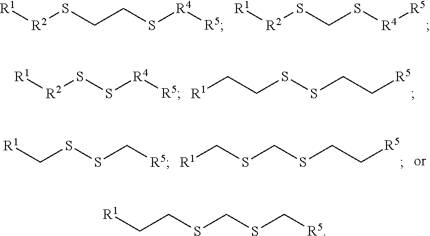

In some examples, including any of the foregoing, R³ is absent or is ethylene. In some examples, including any of the foregoing, R³ is absent. In some examples, including any of the foregoing, R³ is ethylene.

In some examples, including any of the foregoing, the electrolyte additive has the following structure:

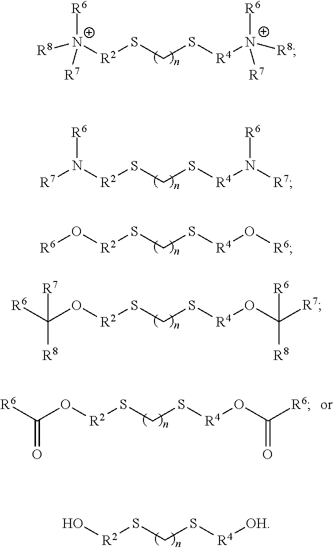

In these examples, subscript n is an integer selected from 0, 1, or 2. In some examples, subscript n is 0. In some examples, subscript n is 1. In some examples, subscript n is 2. In some alkaline electrolytes, the electrolyte additive has the following structure:

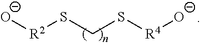

In some examples, including any of the foregoing, the electrolyte additive has the following structure:

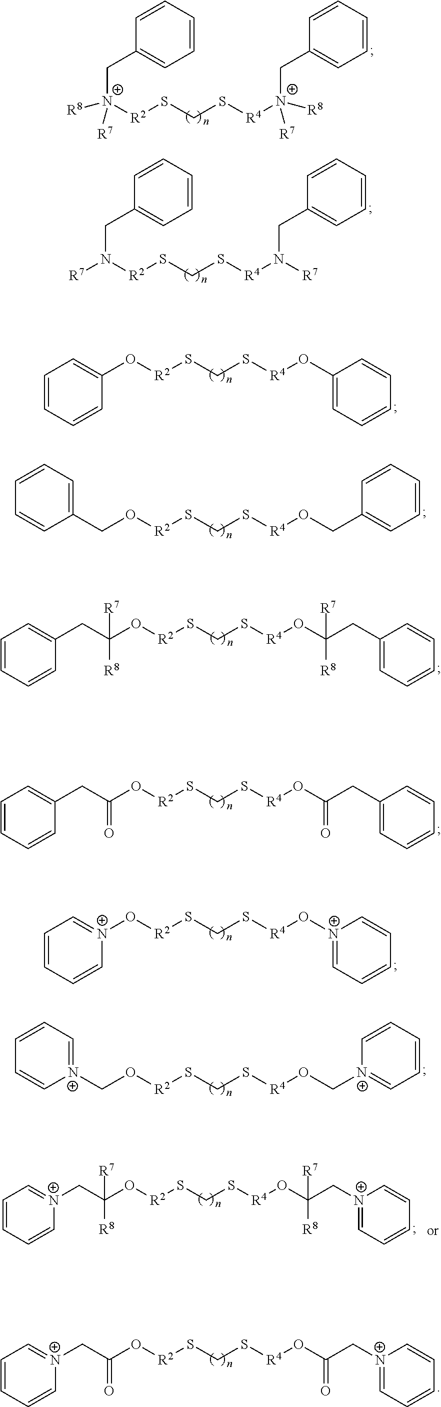

In some of these examples, subscript n is an integer selected from 0, 1, and 2. In some examples, subscript n is 0. In some examples, subscript n is 1. In some examples, subscript n is 2.

In some examples, including any of the foregoing, subscript n is 0, 1, or 2. In some examples, subscript n is 1. In some examples, subscript n is 0. In some examples, subscript n is 2.

In some examples, including any of the foregoing, the electrolyte additive has the following structure:

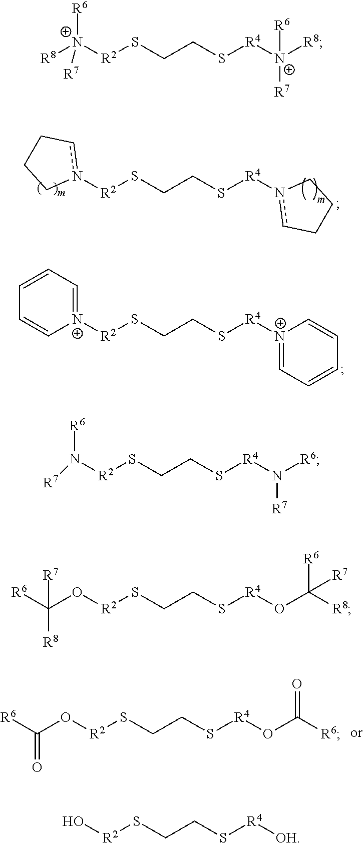

In some examples, subscript m is selected from 0, 1, 2, or 3; and [symbol] is either a single or double bond. In some examples, subscript m is 0. In certain examples, subscript m is 1. In certain examples, subscript m is 2. In certain examples, subscript m is 3. In certain examples, [symbol] is either a single. In certain other examples, [symbol] is a double bond. In some alkaline electrolytes, the electrolyte additive has the following structure:

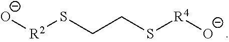

In some examples, including any of the foregoing, the electrolyte additive has the following structure:

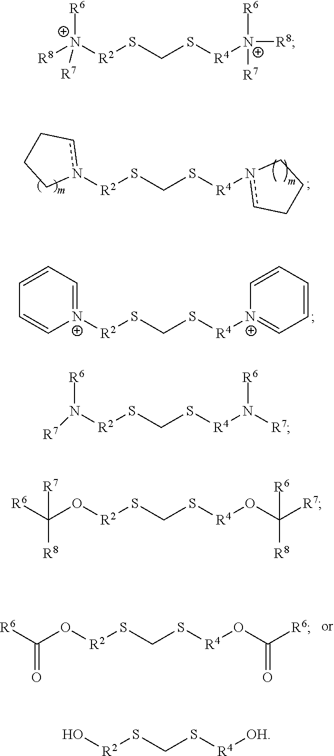

In some examples, subscript m is selected from 0, 1, 2, or 3; and [symbol] is either a single or double bond. In some examples, subscript m is 0. In certain examples, subscript m is 1. In certain examples, subscript m is 2. In certain examples, subscript m is 3. In certain examples, [symbol] is either a single. In certain other examples, [symbol] is a double bond. In some alkaline electrolytes, the electrolyte additive has the following structure:

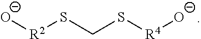

In some examples, including any of the foregoing, the electrolyte additive has the following structure:

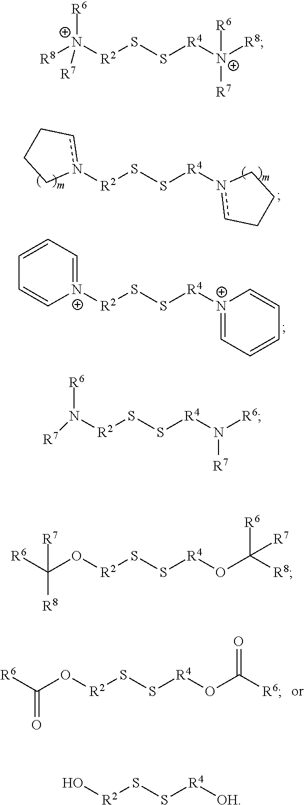

In some examples, subscript m is selected from 0, 1, 2, or 3. In some examples, subscript m is 0. In certain examples, subscript m is 1. In certain examples, subscript m is 2. In certain examples, subscript m is 3. In certain examples, [symbol] is either a single. In certain other examples, [symbol] is a double bond. In some alkaline electrolytes, the electrolyte additive has the following structure:

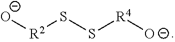

Certain molecules disclosed herein exist in dynamic equilibrium with protonated and de-protonated analogs, in which the equilibrium constant is temperature dependent.

In some examples, including any of the foregoing, R⁶ and R⁷ are both methyl.

In some examples, including any of the foregoing, R⁶ or R⁷ are methyl.

In some examples, including any of the foregoing, R⁶ and R⁷ are both benzyl.

In some examples, including any of the foregoing, R⁶ or R⁷ is benzyl.

In some examples, including any of the foregoing, the electrolyte additive has the following structure:

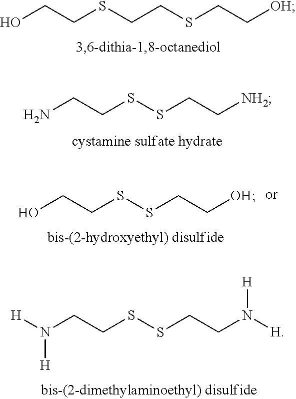

In some alkaline electrolytes, the electrolyte additive has the following structure

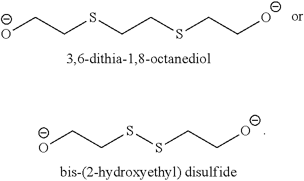 [SMILES: [O-]CCSCCSCC[O-] ; "or" ; [O-]CCSSCC[O-]]

In some examples, the electrolyte is 3,6-dithia-1,8-octanediol

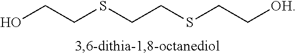

In some examples, the electrolyte is cystamine sulfate hydrate

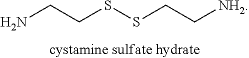

In some examples, the electrolyte is

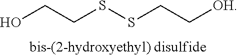

In some examples, the electrolyte is

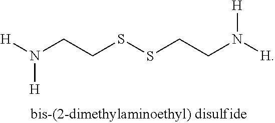

In some examples, the electrolyte is

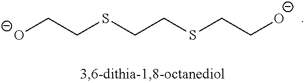

In some examples, the electrolyte is

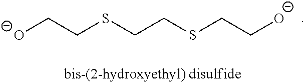

In some electrolytes, the electrolyte additive has the following structure:

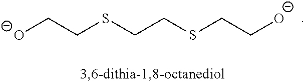

In some electrolytes, the electrolyte additive has the following structure:

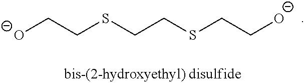

In some examples, including any of the foregoing, the electrolyte additive comprises two or more of the following structures:

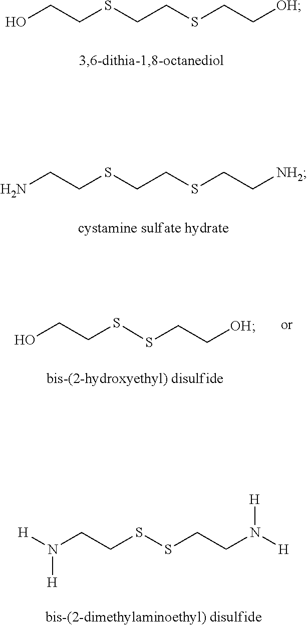 [SMILES: OCCSCCSCCO ; NCCSCCSCCN ; OCCSSCCO ; NCCSSCCN]

In some examples, including any of the foregoing, the electrolyte additive is

In some examples, including any of the foregoing, the electrolyte additive is

In some examples, including any of the foregoing, the electrolyte additive is

In some examples, including any of the foregoing, the electrolyte additive is

In some examples, including any of the foregoing, the electrolyte additive is

In some examples, including any of the foregoing, the electrolyte additive is

In some examples, including any of the foregoing, the electrolyte additive is

In some examples, including any of the foregoing, the electrolyte additive is

In some examples, including any of the foregoing, the electrolyte additive is

In some examples, including any of the foregoing, the electrolyte additive is

In some examples, including any of the foregoing, the electrolyte additive is

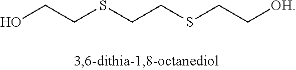

In some examples, including any of the foregoing, the electrolyte additive is

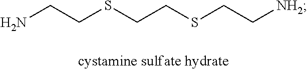

In some examples, including any of the foregoing, the electrolyte additive is

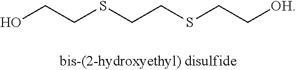

In some examples, including any of the foregoing, the electrolyte additive is.

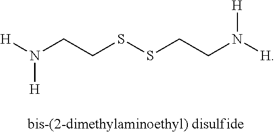

In some examples, including any of the foregoing, the electrolyte additive further includes a counter-ion selected from sulfate, nitrate, chloride, bromide, iodide, or a combination thereof.

In some examples, including any of the foregoing, the electrolyte additive is present in an electrolyte at a concentration equal to, or greater than, 0.01 weight percent (wt %) to less than, or equal to, 25 wt %.

In some examples, including any of the foregoing, the electrolyte additive is present in an electrolyte at a concentration equal to, or greater than, 0.05 weight percent (wt %) to less than, or equal to, 25 wt %.

In some examples, including any of the foregoing, the electrolyte additive is present in an electrolyte at a concentration equal to, or greater than, 0.1 weight percent (wt %) to less than, or equal to, 25 wt %.

In some examples, including any of the foregoing, the electrolyte additive is present in an electrolyte at a concentration equal to, or greater than, 0.5 weight percent (wt %) to less than, or equal to, 25 wt %.

In some examples, including any of the foregoing, the electrolyte additive is present in an electrolyte at a concentration equal to, or greater than, 1 weight percent (wt %) to less than, or equal to, 25 wt %.

In some examples, including any of the foregoing, the electrolyte additive is present in an electrolyte at a concentration equal to, or greater than, 5 weight percent (wt %) to less than, or equal to, 25 wt %.

In some examples, including any of the foregoing, the electrolyte additive is present in an electrolyte at a concentration equal to, or greater than, 10 weight percent (wt %) to less than, or equal to, 25 wt %.

In some examples, including any of the foregoing, the electrolyte additive is present in an electrolyte at a concentration equal to, or greater than, 15 weight percent (wt %) to less than, or equal to, 25 wt %.

In some examples, including any of the foregoing, the electrolyte additive is present in an electrolyte at a concentration equal to, or greater than, 20 weight percent (wt %) to less than, or equal to, 25 wt %.

In some examples, including any of the foregoing, the electrolyte additive is present in an electrolyte at a concentration equal to, or greater than, 0.01 weight percent (wt %) to less than, or equal to, 20 wt %.

In some examples, including any of the foregoing, the electrolyte additive is present in an electrolyte at a concentration equal to, or greater than, 0.01 weight percent (wt %) to less than, or equal to, 15 wt %.

In some examples, including any of the foregoing, the electrolyte additive is present in an electrolyte at a concentration equal to, or greater than, 0.01 weight percent (wt %) to less than, or equal to, 10 wt %.

In some examples, including any of the foregoing, the electrolyte additive is present in an electrolyte at a concentration equal to, or greater than, 0.01 weight percent (wt %) to less than, or equal to, 5 wt %.

In some examples, including any of the foregoing, the electrolyte additive is present in an electrolyte at a concentration equal to, or greater than, 0.01 weight percent (wt %) to less than, or equal to, 1 wt %.

In some examples, including any of the foregoing, the electrolyte additive is present in an electrolyte at a concentration equal to, or greater than, 0.01 weight percent (wt %) to less than, or equal to, 0.5 wt %.

In some examples, including any of the foregoing, the electrolyte additive is present in an electrolyte at a concentration equal to, or greater than, 0.01 weight percent (wt %) to less than, or equal to, 0.1 wt %.

In some examples, including any of the foregoing, the electrolyte additive is present in an electrolyte at a concentration equal to, or greater than, 0.01 weight percent (wt %) to less than, or equal to, 0.05 wt %.

In some examples, including any of the foregoing, the electrolyte additive is present in an electrolyte at a concentration of about 25 weight percent (wt %).

In some examples, including any of the foregoing, the electrolyte additive is present in an electrolyte at a concentration of about 20 weight percent (wt %).

In some examples, including any of the foregoing, the electrolyte additive is present in an electrolyte at a concentration of about 15 weight percent (wt %).

In some examples, including any of the foregoing, the electrolyte additive is present in an electrolyte at a concentration of about 10 weight percent (wt %).

In some examples, including any of the foregoing, the electrolyte additive is present in an electrolyte at a concentration of about 5 weight percent (wt %).

In some examples, including any of the foregoing, the electrolyte additive is present in an electrolyte at a concentration of about 1 weight percent (wt %).

In some examples, including any of the foregoing, the electrolyte additive is present in an electrolyte at a concentration of about 0.5 weight percent (wt %).

In some examples, including any of the foregoing, the electrolyte additive is present in an electrolyte at a concentration of about 0.1 weight percent (wt %).

In some examples, including any of the foregoing, the electrolyte additive is present in an electrolyte at a concentration of about 0.05 weight percent (wt %).

In some examples, including any of the foregoing, the electrolyte additive is present in an electrolyte at a concentration of about 0.01 weight percent (wt %).

In some examples, including any of the foregoing, the electrolyte additive is present in an electrolyte at a concentration of 25 weight percent (wt %).

In some examples, including any of the foregoing, the electrolyte additive is present in an electrolyte at a concentration of 20 weight percent (wt %).

In some examples, including any of the foregoing, the electrolyte additive is present in an electrolyte at a concentration of 15 weight percent (wt %).

In some examples, including any of the foregoing, the electrolyte additive is present in an electrolyte at a concentration of 10 weight percent (wt %).

In some examples, including any of the foregoing, the electrolyte additive is present in an electrolyte at a concentration of 5 weight percent (wt %).

In some examples, including any of the foregoing, the electrolyte additive is present in an electrolyte at a concentration of 1 weight percent (wt %).

In some examples, including any of the foregoing, the electrolyte additive is present in an electrolyte at a concentration of 0.5 weight percent (wt %).

In some examples, including any of the foregoing, the electrolyte additive is present in an electrolyte at a concentration of 0.1 weight percent (wt %).

In some examples, including any of the foregoing, the electrolyte additive is present in an electrolyte at a concentration of 0.05 weight percent (wt %).

In some examples, including any of the foregoing, the electrolyte additive is present in an electrolyte at a concentration of 0.01 weight percent (wt %).

In some examples, including any of the foregoing, the electrolyte additive is present in an electrolyte at a concentration equal to, or greater than, 0.005 weight percent (wt %) to less than, or equal to, 25 wt %.

In some examples, including any of the foregoing, set forth herein is a zinc-battery comprising an electrolyte set forth herein.

The chemical structures set forth herein are generally drawn showing neutrally charged molecules. However, the electrolytes used in many zinc batteries are alkaline and basic. Molecules have labile hydrogen ions (i.e., protons) will exist in a thermodynamic equilibrium; the labile protons will associate and dissociate from the molecule. In basic electrolytes such as those often used with zinc batteries, the aforementioned battery additives may be present in the electrolyte in a deprotonated form. For example, the following electrolyte additives having deprotonated hydroxyl groups may be present in place of, or in addition to, the conjugate acids illustrated above:

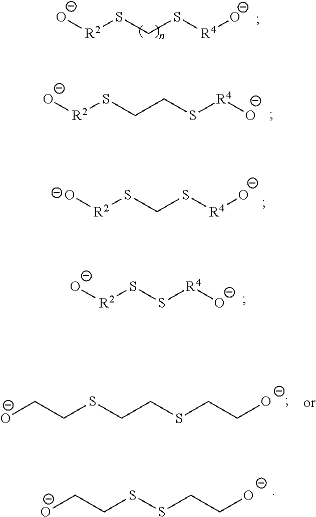

In some examples, the electrolytes used in many zinc batteries are protic and acid. Molecules have labile hydrogen ions (i.e., protons) will exist in a thermodynamic equilibrium; the labile protons will associate and dissociate from the molecule. In acid electrolytes such as those often used with zinc batteries, the aforementioned battery additives may be present in the electrolyte in a protonated form. For example, the following electrolyte additives having ammonium groups may be present in place of, or in addition to, the conjugate bases illustrated above:

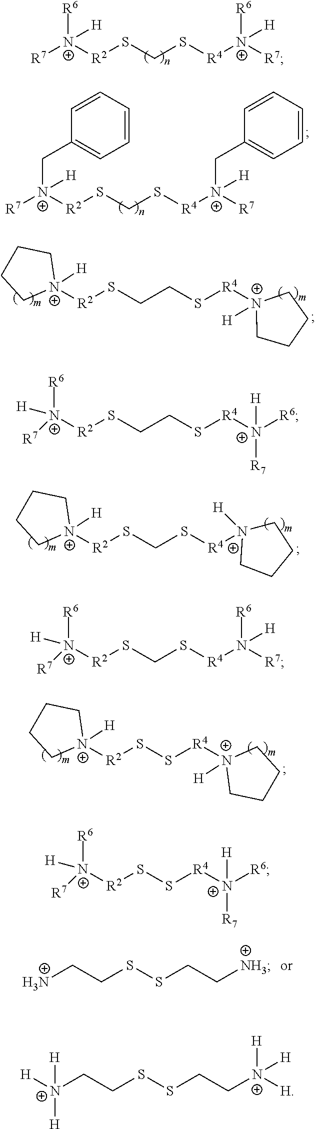

### III. PROCESS FOR MAKING

In some examples, set forth herein is a process for making a zinc battery, comprising contacting an electrolyte set forth herein with a zinc-battery electrode.

In some examples, including any of the foregoing, the zinc-battery comprises a positive electrode.

In some examples, including any of the foregoing, the zinc-battery comprises a negative electrode.

In some examples, including any of the foregoing, the negative electrode is selected from zinc foil, zinc powder, porous zinc, electroplated zinc, zinc alloy, or a combination thereof. In certain examples, the negative electrode is zinc foil. In certain examples, the negative electrode is zinc powder. In certain examples, the negative electrode is porous zinc. In certain other examples, the negative electrode is electroplated zinc. In certain examples, the negative electrode is zinc alloy. In certain examples, the negative electrode is a combination thereof zinc foil, zinc powder, porous zinc, electroplated zinc, and zinc alloy.

### IV. METHODS FOR USING

In some examples, set forth herein is a method of using a zinc battery, comprising electrochemically cycling a zinc-battery comprising an electrolyte set forth herein.

In some examples, set forth herein is a method of using a zinc battery, comprising charging a zinc-battery comprising an electrolyte set forth herein to at least −1 V (relative to Ag/AgCl).

In some examples, including any of the foregoing, the charge current density is less than 10 mA/cm².

In some examples, including any of the foregoing, the charge current density is less than 5 mA/cm².

In some examples, including any of the foregoing, the charge current density is less than 2 mA/cm².

In some examples, including any of the foregoing, the charge current density is at least 0.5 mA/cm².

In some examples, including any of the foregoing, the charge current density is at least 1 mA/cm².

In some examples, including any of the foregoing, the charge current density is at least 50 mA/cm².

In some examples, including any of the foregoing, the charge current density is at least 100 mA/cm².

In some examples, including any of the foregoing, the maximum charge current density is less than 200 mA/cm².

In some examples, including any of the foregoing, the method comprises discharging the zinc-battery.

In some examples, including any of the foregoing, the discharge current density is less than 10 mA/cm².

In some examples, including any of the foregoing, the discharge current density is less than 5 mA/cm².

In some examples, including any of the foregoing, the discharge current density is less than 2 mA/cm².

In some examples, including any of the foregoing, the discharge current density is at least 0.5 mA/cm².

In some examples, including any of the foregoing, the discharge current density is at least 1 mA/cm².

In some examples, including any of the foregoing, the discharge current density is at least 50 mA/cm².

In some examples, including any of the foregoing, the discharge current density is at least 100 mA/cm².

In some examples, including any of the foregoing, the maximum discharge current density is less than 200 mA/cm².

In some examples, including any of the foregoing, the method comprises storing the zinc-battery for at least 1 day.

In some examples, including any of the foregoing, the method comprises discharging the zinc-battery.

In some examples, including any of the foregoing, the zinc-battery demonstrates a Coulombic Efficiency greater than 95% for a charge-discharge cycle.

### V. EXAMPLES

Chemicals were commercially purchased unless stated explicitly otherwise.

Electrochemical cycling was performed on an Princeton Applied Research VersaStat 3 potentiostat.

For Examples 1-3, electrochemical cells were constructed having a negative electrode of either zinc wire or glassy carbon, an aqueous electrolyte, and a platinum counter electrode. Voltages were measured relative to a Ag/AgCl electrode. Unless specified otherwise, the electrolyte included 2 molar (M) ZnBr, 0.5 M KCl, and water. Electrolytes were sparged to remove interfering dissolved gasses by bubbling pure nitrogen gas through them while stirring for 30 to 45 minutes prior to each test. Additional salt concentrations were tested.

For Examples 4-6, electrochemical cells were constructed having a glassy carbon negative electrode, an aqueous electrolyte, and a platinum counter electrode. Voltages were measured relative to a Ag/AgCl electrode. Unless specified otherwise, the electrolyte included 4 molar (M) ZnCl, 3M LiCl and water. Electrolytes were sparged to remove interfering dissolved gasses by bubbling pure nitrogen gas through them while stirring for 30 to 45 minutes prior to each test. Additional salt concentrations were tested.

### Example 1—Control

This Example shows the performance of a zinc battery without an electrolyte additive. An electrochemical cell was constructed as noted above. Zinc was electroplated at the negative electrode at −1 V until a charge capacity of 5.18 mAh was achieved. The electrochemical cell was then discharged at a current density of 100 mA/cm². The cell was observed to have a discharge capacity of 5.06 mAh. This is a Coulombic Efficiency of 97.68%.

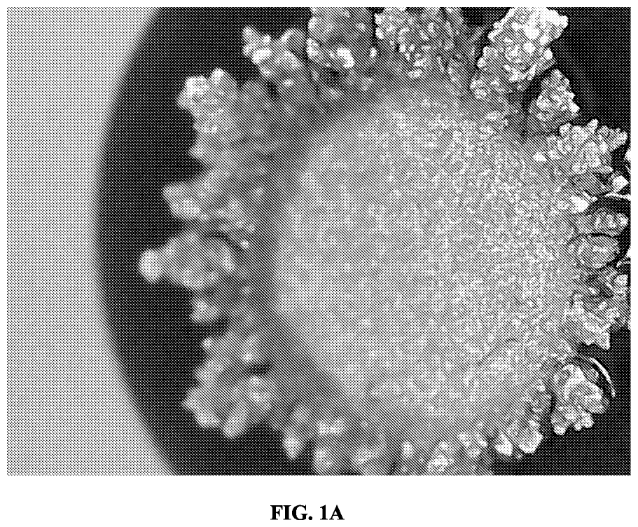

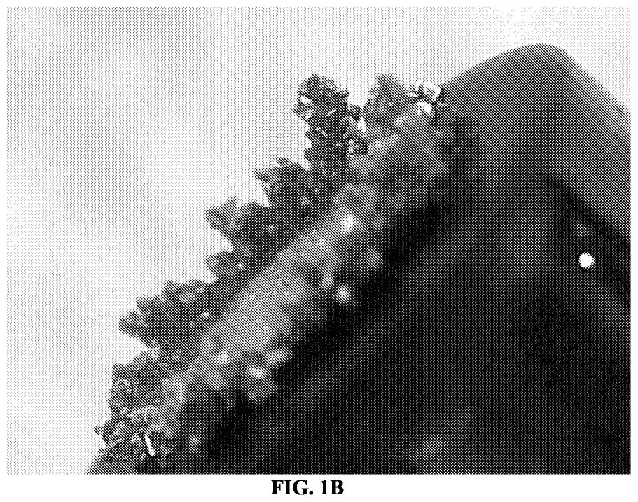

Separately, the electrochemical cell was charged at −1.2 V producing a charge current density of 105 mA/cm². An optical image was taken of the electroplated zinc on the tip of a glassy carbon electrode that is 3 mm in diameter. As shown in FIG. TA and FIG. 1B, zinc dendrite formation is visible and dendrites have grown to lengths of up to 1.5 mm.

This Example demonstrates that without an additive, large dendrite growth is observed. The coulombic efficiency was characterized as good.

### Example 2—3,6 Dithia-1,8-octanediol

This Example shows the performance of a zinc battery. An electrochemical cell was constructed as noted above but with 1 wt % of 3,6-dithia-1,8-octanediol as an electrolyte additive. Zinc was electroplated at the negative electrode at −1 V until a charge capacity of 3.22 mAh was achieved. The electrochemical cell was then discharged at a current density of 100 mA/cm². The cell was observed to have a discharge capacity of 3.15 mAh. This is a Coulombic Efficiency of 97.66%.

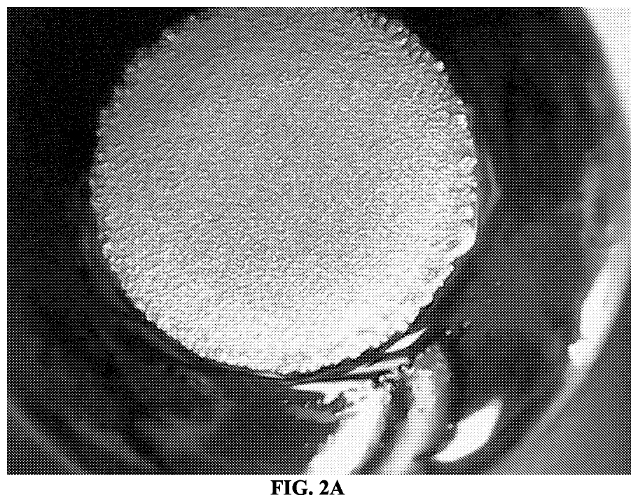

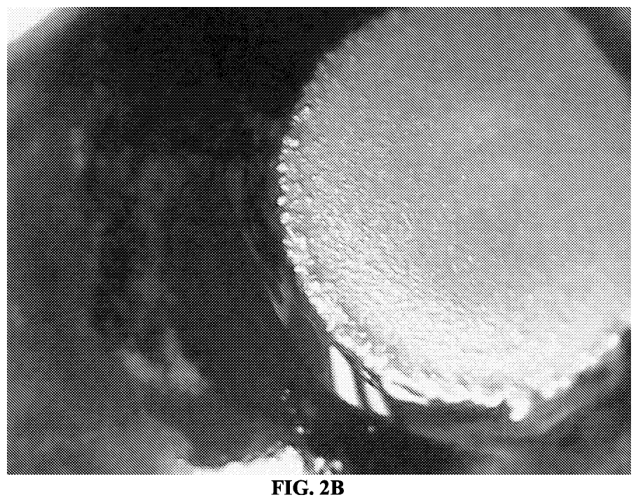

Separately, the electrochemical cell was charged at −1.2 V and at a charge current density of 81 mA/cm². An optical image was taken of the electroplated zinc on the tip of a glassy carbon electrode that is 3 mm in diameter. As shown in FIG. 2A and FIG. 2B, zinc dendrite formation is dramatically less than that of FIG. TA and FIG. 1B. The largest dendrites grown in this Example measure less than 0.2 mm, representing a reduction of 86% or more from Example 1.

It is possible to evaluate the generation of hydrogen as a source of battery performance degradation. Methods for doing so are set forth in US 2020/0243909 A1, titled ZINC BATTERY ELECTROLYTE ADDITIVE, which published Jul. 30, 2020, and is assigned to Octet Scientific, Inc., the entire contents of which are herein incorporated by reference in its entirety for all purposes.

### Example 3—Bis(2-dimethylaminoethyl) disulfide dihydrochloride

This Example shows the performance of a zinc battery. An electrochemical cell was constructed as noted above but with 0.5 wt % of Bis(2-dimethylaminoethyl) disulfide dihydrochloride as an electrolyte additive. Zinc was electroplated at the negative electrode at −1 V until a charge capacity of 3.68 mAh was achieved. The electrochemical cell was then discharged at a current density of 100 mA/cm². The cell was observed to have a discharge capacity of 3.51 mAh. This is a Coulombic Efficiency of 95.46%.

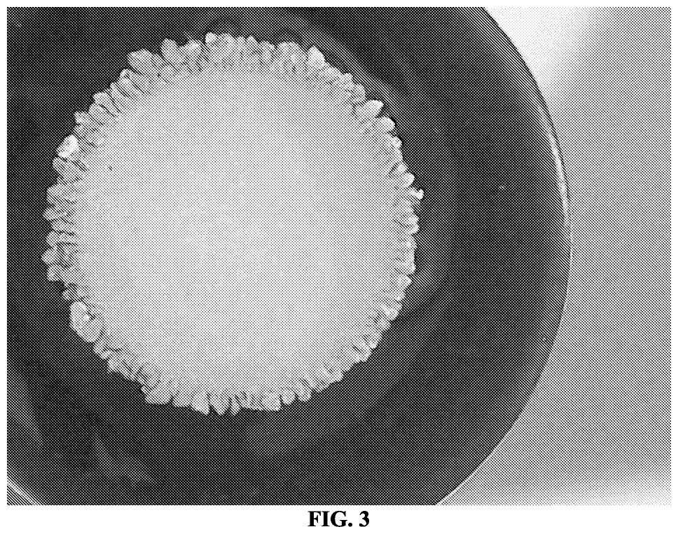

Separately, the electrochemical cell was charged at −1.2 V producing a charge current density of 71 mA/cm². An optical image was taken of the electroplated zinc on the tip of a glassy carbon electrode that is 3 mm in diameter. As shown in FIG. 3, zinc dendrite dramatically less than that of FIG. 1A and FIG. 1B. The largest dendrites from in this Example measure less than 0.4 mm, representing a reduction of 73% or more from Example 1.

### Example 4—Control

This Example shows the performance of a zinc battery without an electrolyte additive. An electrochemical cell was constructed as noted above. Zinc was electroplated at the negative electrode at −20 mA/cm² until a charge capacity of 1.20 mAh was achieved. The electrochemical cell was then discharged at a current density of 20 mA/cm². The cell was observed to have a discharge capacity of 1.09 mAh. This is a Coulombic Efficiency of 90.83%.

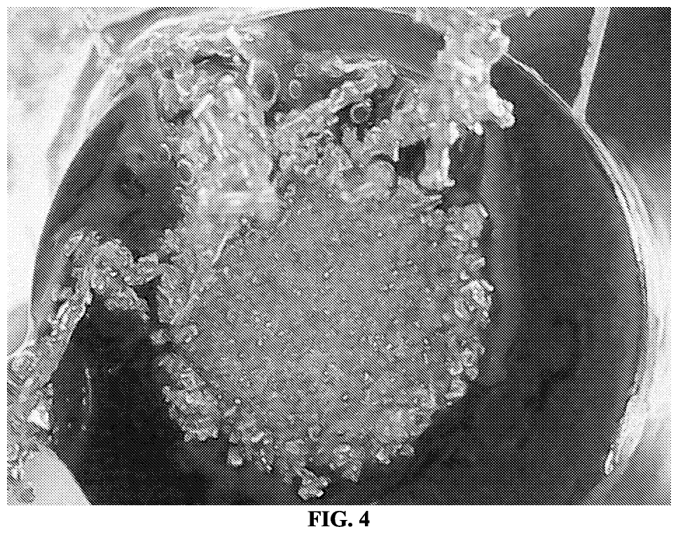

Separately, the electrochemical cell was charged at −0.95 V and at a charge current density of 90 mA/cm². An optical image was taken of the electroplated zinc on the tip of a glassy carbon electrode that is 3 mm in diameter. As shown in FIG. 4, zinc dendrite formation is visible and dendrites have grown to lengths of greater than 2 mm.

This Example demonstrates that without an additive, large dendrite growth is observed. The coulombic efficiency was characterized as good.

### Example 5—3,6 Dithia-1,8-octanediol

This Example shows the performance of a zinc battery. An electrochemical cell was constructed as noted above but with 1 wt % of 3,6-dithia-1,8-octanediol as an electrolyte additive. Zinc was electroplated at the negative electrode at −20 mA/cm² until a charge capacity of 1.20 mAh was achieved. The electrochemical cell was then discharged at a current density of 20 mA/cm². The cell was observed to have a discharge capacity of 1.10 mAh. This is a Coulombic Efficiency of 91.67%.

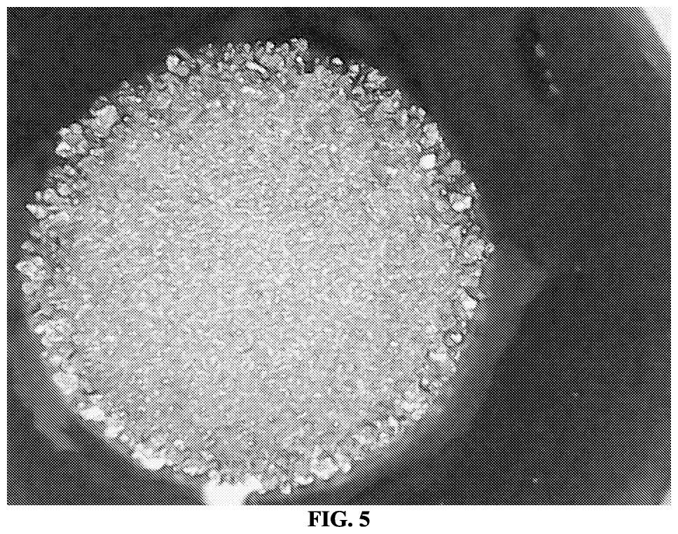

Separately, the electrochemical cell was charged at −0.95 V and at a charge current density of 83 mA/cm². An optical image was taken of the electroplated zinc on the tip of a glassy carbon electrode that is 3 mm in diameter. As shown in FIG. 5, zinc dendrite dramatically less than that of FIG. 4. The largest dendrites from in this Example measure less than 0.4 mm, representing a reduction of 80% or more from Example 4.

### Example 6—Cystamine Dihydrochloride

This Example shows the performance of a zinc battery. An electrochemical cell was constructed as noted above but with 1 wt % of cystamine dihydrochloride as an electrolyte additive. Zinc was electroplated at the negative electrode at −20 mA/cm² until a charge capacity of 1.20 mAh was achieved. The electrochemical cell was then discharged at a current density of 20 mA/cm². The cell was observed to have a discharge capacity of 0.74 mAh. This is a Coulombic Efficiency of 61.97%.

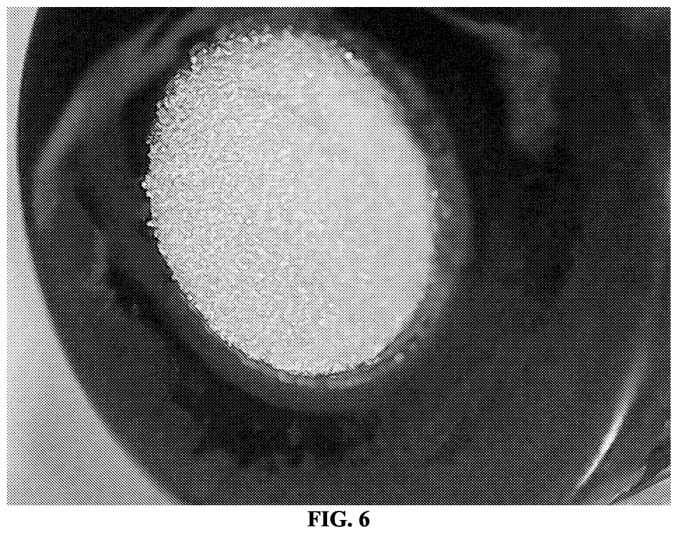

Separately, the electrochemical cell was charged at −0.95 V and at a charge current density of 62 mA/cm². An optical image was taken of the electroplated zinc on the tip of a glassy carbon electrode that is 3 mm in diameter. As shown in FIG. 6, zinc dendrite dramatically less than that of FIG. 4. The largest dendrites from in this Example measure less than 0.2 mm, representing a reduction of 90% or more from Example 4.

The embodiments and examples described above are intended to be merely illustrative and non-limiting. Those skilled in the art will recognize or will be able to ascertain using no more than routine experimentation, numerous equivalents of specific compounds, materials and procedures. All such equivalents are considered to be within the scope and are encompassed by the appended claims.

What is claimed is:

1. An electrolyte additive, having the following structure: 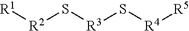
wherein:
R¹ and R⁵ are, independently in each instance, selected from the group consisting of —OH, —NR⁶R⁷; —NR⁶R⁷R⁸; —O—R⁶; —O—CR⁶R⁷R⁸; —O—C(O)—R⁸; and —C(O)OH;
R² and R⁴ are, independently in each instance, selected from the group consisting of an optionally substituted alkylene and an optionally substituted heteroalkylene, wherein the alkylene and heteroalkylene, independently in each instance, has 1 to 10 carbon atoms;
R³ is absent or is selected from the group consisting of an optionally substituted alkylene and an optionally substituted heteroalkylene, wherein the alkylene and heteroalkylene, independently in each instance, has 1 to 10 carbon atoms;
R⁶ and R⁷ are, independently in each instance, selected from the group consisting of H, alkyl, heteroalkyl, aryl, heteroaryl, alkyl-aryl, and alkyl-heteroaryl; wherein the alkyl, heteroalkyl, aryl, heteroaryl, alkyl-aryl, and alkyl-heteroaryl are unsubstituted; or
R⁶ and R⁷, together with the atom to which they are attached, form an optionally substituted cycloalkyl or heterocycloalkyl having 4 to 6 carbon atoms;
R⁸ is absent or is selected from the group consisting of H, alkyl, heteroalkyl, aryl, heteroaryl, alkyl-aryl, and alkyl-heteroaryl; wherein the alkyl, heteroalkyl, aryl, heteroaryl, alkyl-aryl, and alkyl-heteroaryl are unsubstituted;
wherein electrolyte additive is present in an electrolyte at a concentration equal to, or greater than, 0.005 weight percent (wt %) to less than, or equal to, 25 wt %.

2. The electrolyte additive of claim 1, having the following structure:

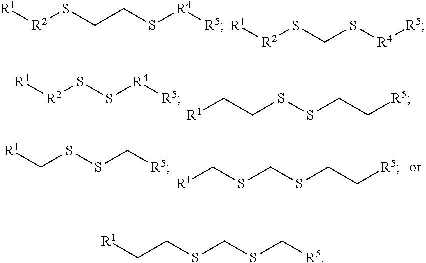

3. The electrolyte additive of claim 1, wherein R³ is absent or is ethylene.

4. The electrolyte additive of claim 1, having the following structure: 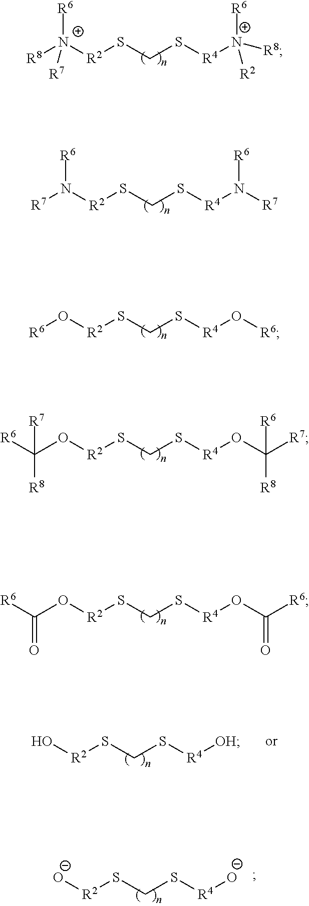
wherein subscript n is an integer selected from 0, 1, or 2.

5. The electrolyte additive of claim 1 or 4, having the following structure: 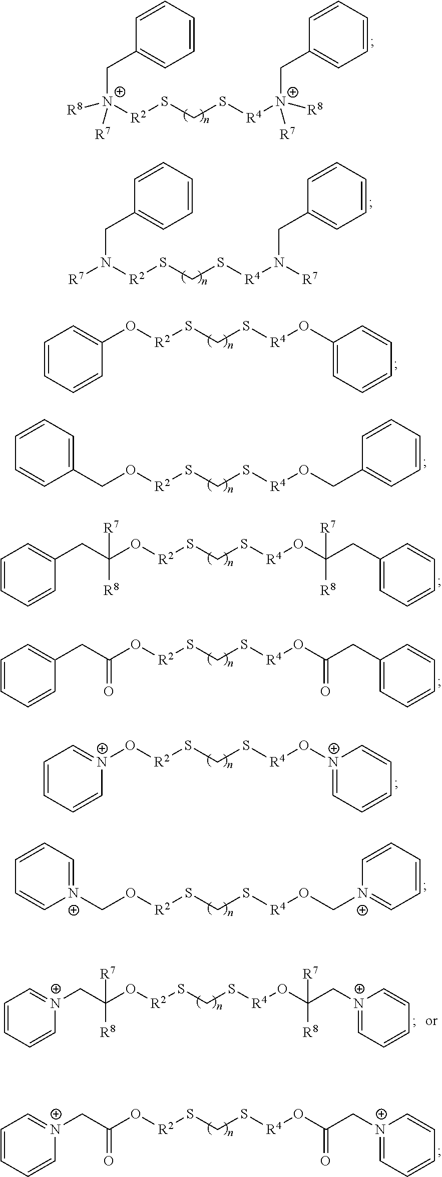
wherein subscript n is an integer selected from 0, 1, or 2.

6. The electrolyte additive of any one of claims 1-5, wherein n is 0, 1, or 2.

7. The electrolyte additive of claim 1, having the following structure: 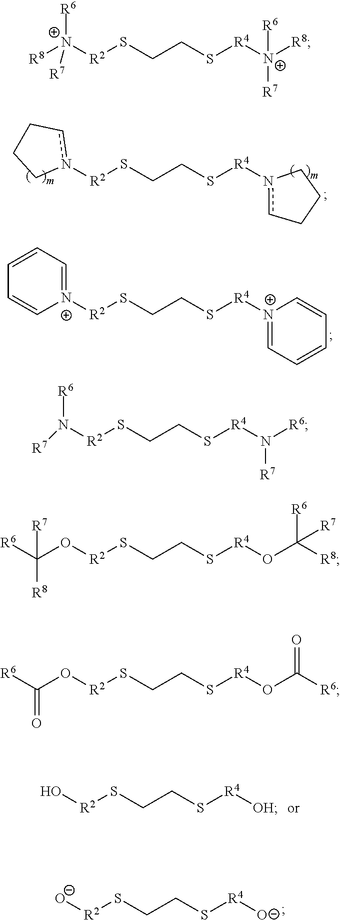
wherein subscript m is selected from 0, 1, 2, or 3; and [symbol] is either a single or double bond.

8. The electrolyte additive of claim 1, having the following structure: 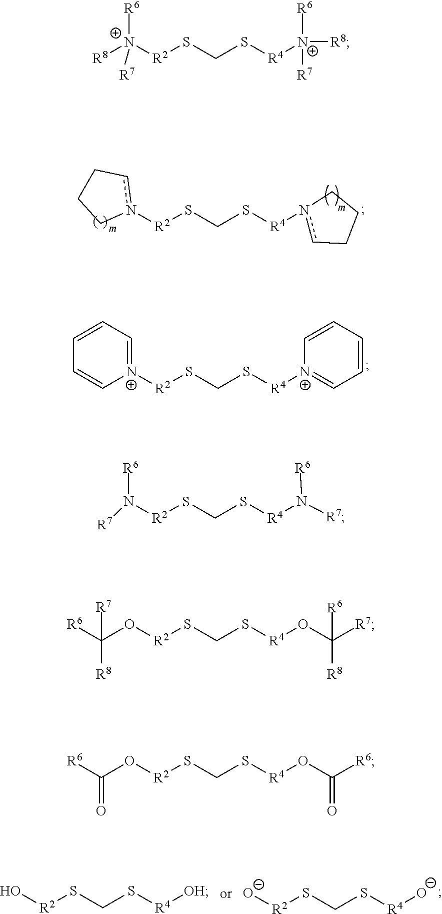
wherein subscript m is selected from 0, 1, 2, or 3; and [symbol] is either a single or double bond.

9. The electrolyte additive of claim 1, having the following structure: 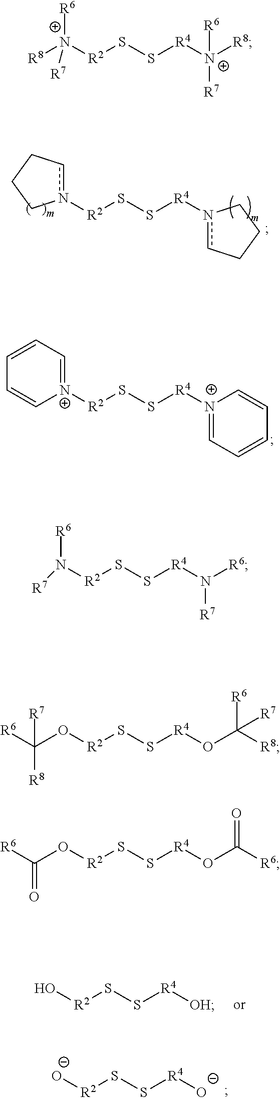
wherein subscript m is selected from 0, 1, 2, or 3; and [symbol] is either a single or double bond.

10. The electrolyte additive of any one of claims 1-9, wherein R⁶ and R⁷ are both methyl.

11. The electrolyte additive of any one of claims 1-9, wherein R⁶ or R⁷ is benzyl.

12. The electrolyte additive of claim 1, having the following structure:

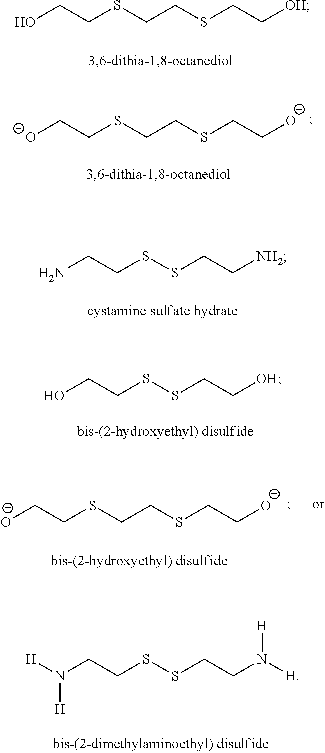

13. The electrolyte additive of claim 1, comprising at least two or more of the following structures:

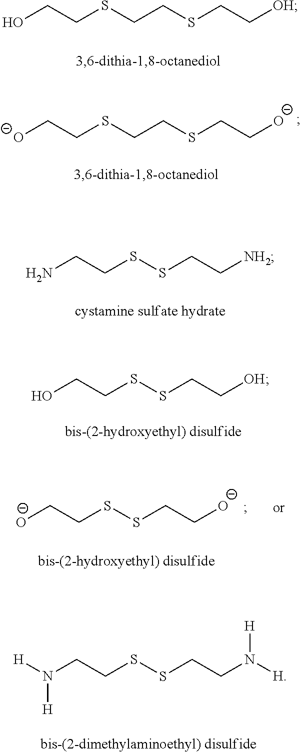

14. The electrolyte additive of any one of claims 1-13, further comprising a counter-ion selected from sulfate, nitrate, chloride, bromide, iodide, or a combination thereof.

15. The electrolyte additive of any one of claims 1-14, wherein electrolyte additive is present in an electrolyte at a concentration equal to, or greater than, 0.01 weight percent (wt %) to less than, or equal to, 25 wt %.

16. The electrolyte additive of any one of claims 1-14, wherein electrolyte additive is present in an electrolyte at a concentration equal to, or greater than, 0.05 weight percent (wt %) to less than, or equal to, 25 wt %.

17. The electrolyte additive of any one of claims 1-14, wherein electrolyte additive is present in an electrolyte at a concentration equal to, or greater than, 0.1 weight percent (wt %) to less than, or equal to, 25 wt %.

18. The electrolyte additive of any one of claims 1-14, wherein electrolyte additive is present in an electrolyte at a concentration equal to, or greater than, 0.5 weight percent (wt %) to less than, or equal to, 25 wt %.

19. The electrolyte additive of any one of claims 1-14, wherein electrolyte additive is present in an electrolyte at a concentration equal to, or greater than, 1 weight percent (wt %) to less than, or equal to, 25 wt %.

20. The electrolyte additive of any one of claims 1-14, wherein electrolyte additive is present in an electrolyte at a concentration equal to, or greater than, 5 weight percent (wt %) to less than, or equal to, 25 wt %.

21. The electrolyte additive of any one of claims 1-14, wherein electrolyte additive is present in an electrolyte at a concentration equal to, or greater than, 10 weight percent (wt %) to less than, or equal to, 25 wt %.

22. The electrolyte additive of any one of claims 1-14, wherein electrolyte additive is present in an electrolyte at a concentration equal to, or greater than, 15 weight percent (wt %) to less than, or equal to, 25 wt %.

23. The electrolyte additive of any one of claims 1-14, wherein electrolyte additive is present in an electrolyte at a concentration equal to, or greater than, 20 weight percent (wt %) to less than, or equal to, 25 wt %.

24. The electrolyte additive of any one of claims 1-14, wherein electrolyte additive is present in an electrolyte at a concentration equal to, or greater than, 0.01 weight percent (wt %) to less than, or equal to, 20 wt %.

25. The electrolyte additive of any one of claims 1-14, wherein electrolyte additive is present in an electrolyte at a concentration equal to, or greater than, 0.01 weight percent (wt %) to less than, or equal to, 15 wt %.

26. The electrolyte additive of any one of claims 1-14, wherein electrolyte additive is present in an electrolyte at a concentration equal to, or greater than, 0.01 weight percent (wt %) to less than, or equal to, 10 wt %.

27. The electrolyte additive of any one of claims 1-14, wherein electrolyte additive is present in an electrolyte at a concentration equal to, or greater than, 0.01 weight percent (wt %) to less than, or equal to, 5 wt %.

28. The electrolyte additive of any one of claims 1-14, wherein electrolyte additive is present in an electrolyte at a concentration equal to, or greater than, 0.01 weight percent (wt %) to less than, or equal to, 1 wt %.

29. The electrolyte additive of any one of claims 1-14, wherein electrolyte additive is present in an electrolyte at a concentration equal to, or greater than, 0.01 weight percent (wt %) to less than, or equal to, 0.5 wt %.

30. The electrolyte additive of any one of claims 1-14, wherein electrolyte additive is present in an electrolyte at a concentration equal to, or greater than, 0.01 weight percent (wt %) to less than, or equal to, 0.1 wt %.

31. The electrolyte additive of any one of claims 1-14, wherein electrolyte additive is present in an electrolyte at a concentration equal to, or greater than, 0.01 weight percent (wt %) to less than, or equal to, 0.05 wt %.

32. The electrolyte additive of any one of claims 1-14, wherein electrolyte additive is present in an electrolyte at a concentration of 25 weight percent (wt %).

33. The electrolyte additive of any one of claims 1-14, wherein electrolyte additive is present in an electrolyte at a concentration of 20 weight percent (wt %).

34. The electrolyte additive of any one of claims 1-14, wherein electrolyte additive is present in an electrolyte at a concentration of 15 weight percent (wt %).

35. The electrolyte additive of any one of claims 1-14, wherein electrolyte additive is present in an electrolyte at a concentration of 10 weight percent (wt %).

36. The electrolyte additive of any one of claims 1-14, wherein electrolyte additive is present in an electrolyte at a concentration of 5 weight percent (wt %).

37. The electrolyte additive of any one of claims 1-14, wherein electrolyte additive is present in an electrolyte at a concentration of 1 weight percent (wt %).

38. The electrolyte additive of any one of claims 1-14, wherein electrolyte additive is present in an electrolyte at a concentration of 0.5 weight percent (wt %).

39. The electrolyte additive of any one of claims 1-14, wherein electrolyte additive is present in an electrolyte at a concentration of 0.1 weight percent (wt %).

40. The electrolyte additive of any one of claims 1-14, wherein electrolyte additive is present in an electrolyte at a concentration of 0.05 weight percent (wt %).

41. The electrolyte additive of any one of claims 1-14, wherein electrolyte additive is present in an electrolyte at a concentration of 0.01 weight percent (wt %).

42. The electrolyte additive of any one of claims 1-14, wherein electrolyte additive is present in an electrolyte at a concentration equal to, or greater than, 0.005 weight percent (wt %) to less than, or equal to, 25 wt %.

43. A zinc-battery comprising the electrolyte additive of any one of claims 1-42.

44. A process for making a zinc battery, comprising
contacting an electrolyte comprising the electrolyte additive of any one of claims 1-14 with a zinc-battery electrode.

45. The process of claim 44, wherein the zinc-battery comprises a positive electrode.

46. The process of claim 44, wherein the zinc-battery comprises a negative electrode.

47. The process of claim 46, wherein the negative electrode is selected from zinc foil, zinc powder, porous zinc, electroplated zinc, zinc alloy, or a combination thereof.

48. A method of using a zinc battery, comprising electrochemically cycling a zinc-battery comprising the electrolyte additive of any one of claims 1-42.

49. A method of using a zinc battery, comprising charging a zinc-battery comprising the electrolyte additive of any one of claims 1-42 to at least −1 V (relative to Ag/AgCl).

50. The method of claim 49, wherein the charge current density is less than 10 mA/cm².

51. The method of claim 49, wherein the charge current density is less than 5 mA/cm².

52. The method of claim 49, wherein the charge current density is less than 2 mA/cm².

53. The method of any one of claims 50-52, wherein the charge current density is at least 0.5 mA/cm².

54. The method of any one of claims 50-52, wherein the charge current density is at least 1 mA/cm².

55. The method of claim 49, wherein the charge current density is at least 50 mA/cm².

56. The method of claim 49 or 50, wherein the charge current density is at least 100 mA/cm².

57. The method of claim 55 or 56, wherein the maximum charge current density is less than 200 mA/cm².

58. The method of any one of claims 49-57, further comprising discharging the zinc-battery.

59. The method of claim 58, wherein the discharge current density is less than 10 mA/cm².

60. The method of claim 58, wherein the discharge current density is less than 5 mA/cm².

61. The method of claim 58, wherein the discharge current density is less than 2 mA/cm².

62. The method of any one of claims 59-61, wherein the discharge current density is at least 0.5 mA/cm².

63. The method of any one of claims 59-61, wherein the discharge current density is at least 1 mA/cm².

64. The method of claim 63, wherein the discharge current density is at least 50 mA/cm².

65. The method of claim 49 or 64, wherein the discharge current density is at least 100 mA/cm².

66. The method of claim 64 or 65, wherein the maximum discharge current density is less than 200 mA/cm².

67. The method of any one of claims 49-66, comprising storing the zinc-battery for at least 1 day.

68. The method of claim 67, further comprising discharging the zinc-battery.

69. The method of any one of claims 49-68, wherein the zinc-battery demonstrates a Coulombic Efficiency greater than 95% for a charge-discharge cycle.
